(19) United States
(12) Patent Application Publication
(10) Pub. No.: US 2010/0100080 A1
(43) Pub. Date: Apr. 22, 2010

(54) System and method for voice activation of surgical instruments

(76) Inventors: John C. Huculak, Mission Viejo, CA (US); Frederick M. Reed, Cypress, CA (US); Qiuling Zhan, Irvine, CA (US)

Correspondence Address: ALCON, IP LEGAL, TB4-8, 6201 SOUTH FREEWAY, FORT WORTH, TX, 76134, US

(21) Appl. No.: 12/573,554

(22) Filed: Oct. 5, 2009

Related U.S. Application Data
(60) Provisional application No. 61/106,015, filed on Oct. 16, 2008.

(51) Int. Cl. A61B 17/00

(52) U.S. Cl. 606/1

(57) ABSTRACT

The present invention provides a system and method for operating a surgical console and/or a device attached to the surgical console, and embodiment of the method comprising the steps of: enabling a voice command channel at the surgical console; receiving a voice command trigger; receiving a voice command; confirming the voice command; and causing the surgical console to execute one or more actions associated with the voice command. The surgical console may include a microprocessor, memory, procedural recorder, a user interface and interface(s) through which peripheral devices couple to the console. The microprocessor may direct operations of the surgical console and of peripheral devices that couple to the surgical console. The memory in addition to containing instructions which the microprocessor uses to direct the operation of the surgical console and peripheral devices may also store recorded surgical procedures. The user interface allows users to initialize and control operation of the surgical console via spoken commands. The surgical console can comprise a voice command module operably coupled to the user interface and the microprocessor for processing voice commands and causing the surgical console or a peripheral device to execute one or more functions associated with the voice command.

This application claims priority to U.S. Provisional Application Ser. No. 61/106,015 filed Oct. 16, 2008.

### TECHNICAL FIELD OF THE INVENTION

The present invention relates generally to surgical consoles systems and methods, and more particularly, to a system and method for voice activation of a surgical console and surgical instruments associated with the surgical console.

### BACKGROUND OF THE INVENTION

During modern surgery, particularly ophthalmic surgery, the surgeon uses a variety of pneumatic and electronically driven microsurgical handpieces. The handpieces are operated by a microprocessor-driven surgical console that receives inputs from the surgeon or an assistant by a variety of peripheral devices, such as foot pedal controllers, infrared remote control devices and menu-driven touch screens. One such microsurgical console is described in U.S. Pat. No. 5,455,766 (Scheller, et al.), the entire content of which is incorporated herein by reference. Surgical consoles allow surgeons to manually input surgical operating parameters, select surgical handpieces and otherwise control the operation of the surgical console and of device attached to the surgical console. However, these prior art surgical consoles require that operating parameters and methodologies be inputted manually using a keypad, touch screen or downloaded from another console that has had the parameters inputted manually and likewise require physical contact to control the various operations of the console, such as by touching buttons on a control panel or touch screen, operating a remote control or by stepping on a footswitch.

This type of physical contact, however, typically requires crossing the sterile surgical barrier if a sterile person attempts to control the surgical console or attached devices via the surgical console. For example, touching buttons on the surgical console will likely require crossing the sterile barrier or otherwise require the availability of a non-sterile assistant. Often, a non-sterile assistant is not readily available, causing delays in the surgical procedure and crossing the sterile barrier is undesirable as it puts sterile maintenance at risk. Further, touching remote control buttons inside the sterile barrier typically requires the surgeon to switch his or her gaze from looking through the surgical microscope to the location of the remote control unit.

Accordingly, a need exists for a method and system for voice activation and control of a surgical console and associated surgical instrumentation or devices connected to the surgical console without the disadvantages of prior art surgical console control systems and methods.

### SUMMARY OF THE INVENTION

The present invention provides a system and method for voice activation and control of a surgical console and of devices connected to the surgical console. This system and method substantially addresses the above-identified needs, as well as others. More specifically, the present invention provides, in a first embodiment, a method of operating a surgical console and/or a device attached to the surgical console comprising the steps of: enabling a voice command channel at the surgical console; receiving a voice command trigger; receiving a voice command; confirming the voice command; and causing the surgical console to execute one or more actions associated with the voice command.

Another embodiment provides a surgical console operable to control operation of a surgical console function via a voice command. This surgical console may include a microprocessor, memory, procedural recorder, a user interface and interface(s) through which peripheral devices couple to the console. The microprocessor may direct operations of the surgical console and of peripheral devices that couple to the surgical console. The memory in addition to containing instructions which the microprocessor uses to direct the operation of the surgical console and peripheral devices may also store recorded surgical procedures. The user interface allows users or operators to initialize and control operation of the surgical console via a spoken command. The surgical console can comprise a voice command module operably coupled to the user interface and the microprocessor for processing voice commands and causing the surgical console or a peripheral device to execute one or more functions associated with the voice command. The surgical console user interface can further comprise a user feedback device, such as an audible or visual indicator, to generate a command confirmation signal recognizable by the user

The present invention improves upon the prior art by providing a surgical console that can be controlled using audible (spoken voice) commands. Further, peripheral devices such as surgical instruments connected to the surgical console can also be activated and controlled via an audible command. Additionally, embodiments of the method and system of this invention can be implemented in surgical consoles such as, but not limited to, the SERIES TWENTY THOUSAND® LEGACY® surgical system, the ACCURUS® surgical system, and the INFINITY® surgical system, all available from Alcon Laboratories, Inc. This allows the operator or surgeon to operate and control the surgical console and connected peripheral devices without violating the integrity of the sterile surgical field and/or without having to shift attention from viewing the surgical site to a control panel. Embodiments of this invention can also be incorporated within other surgical machines or systems for use in ophthalmic or other surgery, as will be known to those having skill in the art. Other uses for a system and method for voice-activation and control of a surgical console and associated devices in accordance with the teachings of this invention will be known to those having skill in the art.

Accordingly, embodiments of the present invention provide a system and method for voice activation and control of a surgical console and of devices connected to the surgical console that can be implemented in ophthalmic and is other surgical systems and machines to provide accurate voice command driven control of the system or machine without the need for violating sterility of a surgical site and without the distractions associated with a separate visual and/or tactile control panel. These and other advantages and objectives of the present invention will become apparent from the detailed description and claims that follow.

### BRIEF DESCRIPTION OF THE DRAWINGS

For a more complete understanding of the present invention and the advantages thereof, reference is now made to the following description taken in conjunction with the accompanying drawings in which like reference numerals indicate like features and wherein:

FIG. 1 is a perspective view of one surgical console that may be used with embodiments of the present invention;

FIG. 2 is a perspective view of another surgical console that may be used with embodiments of the present invention;

FIGS. 3A, 3B and 3C are a flow chart indicating the steps of one embodiment of the present invention; and

FIGS. 4A, 4B and 4C are a flow chart indicating the steps of another embodiment of the present invention.

### DETAILED DESCRIPTION OF THE INVENTION

Preferred embodiments of the present invention are illustrated in the FIGURES, like numerals being used to refer to like and corresponding parts of the various drawings.

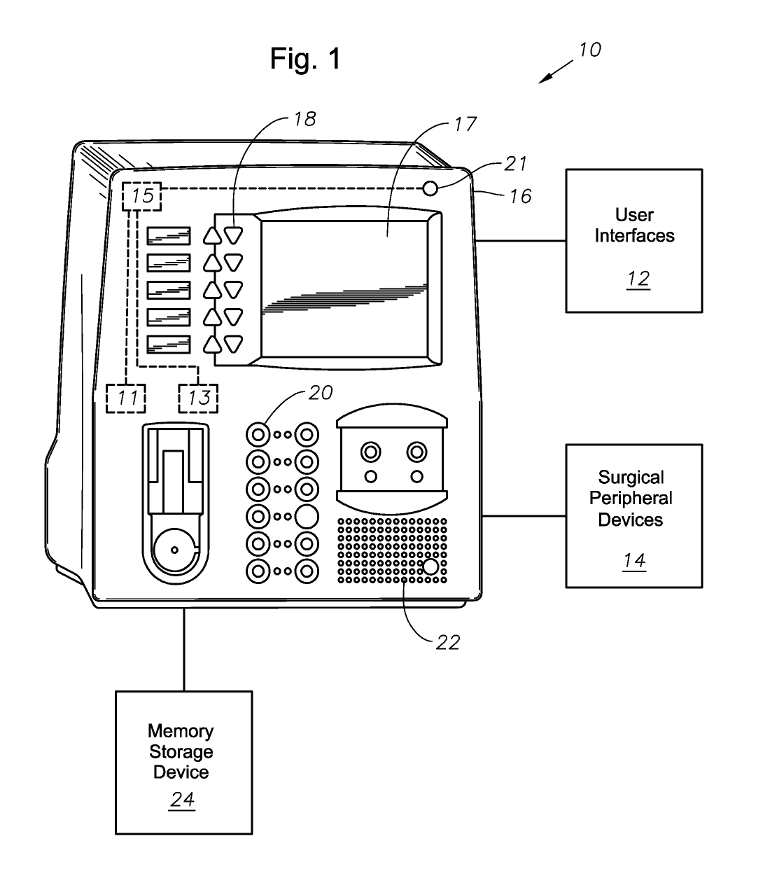

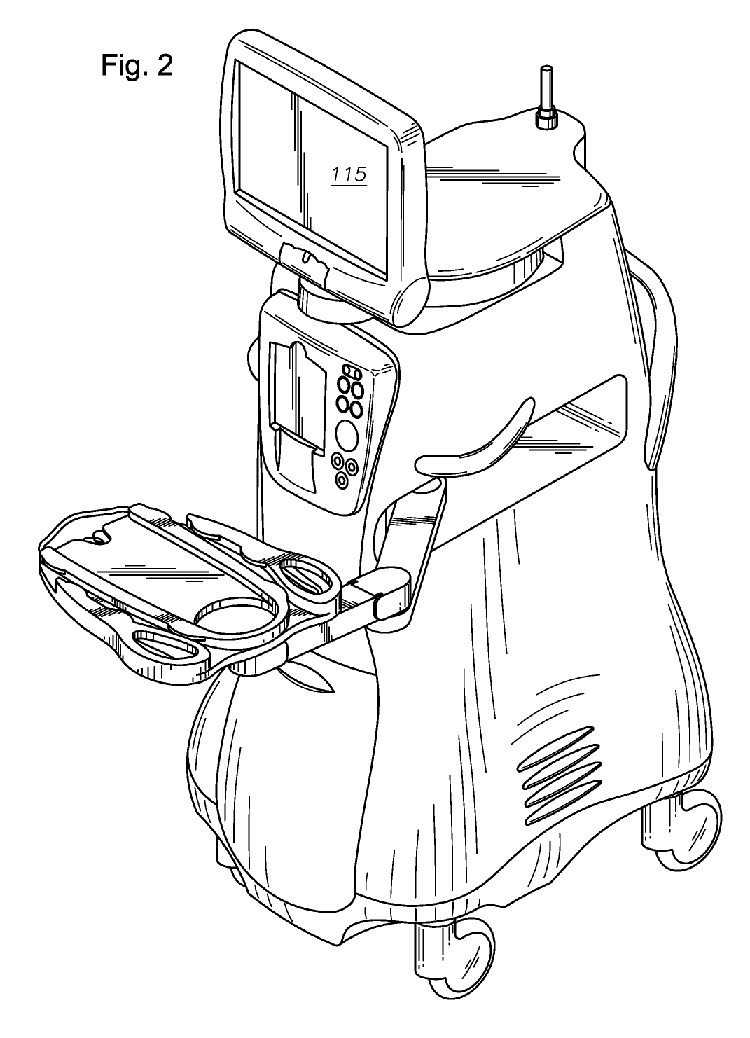

The system and method for voice activation and control of a surgical console provided by embodiments of the present invention may be used with any suitable surgical console, such as, but not limited to, the SERIES TWENTY THOUSAND® LEGACY®, the INFINITI® or the ACCURUS® surgical system consoles, as seen in FIGS. 1 and 2, all commercially available from Alcon Laboratories, Inc., Fort Worth, Tex.

FIG. 1 provides an illustration of surgical console 10. Surgical console 10 has user interfaces 12 and may couple to peripheral devices 14, such as a foot pedal assembly or other push-button type assembly not shown. Console 10 allows an operator, such as a surgeon, to begin a surgical procedure by setting the initial operating parameters and modes into the console. This may be done by allowing the operator to interface with the surgical console through user interfaces 12 or other interfaces provided on front panel 16. These may include an electronic display screen 17, a plurality of push-button switches or touch-sensitive pads 18, the plurality of endless digital potentiometer knobs 20, a microphone 21 or other like interfaces known to those skilled in the art. Push-buttons 18 and knobs 20 are actuable by an operator to access various different operating modes and functions used in various surgical parameters or console functions. Console 10 may also include the ability to accept storage media such as cassette tapes, memory cards, floppy disks, or other like devices known to those skilled in the art.

Electronic display screen 17 may be controlled by a microprocessor that allows the operator access to one or more different menus or messages which relate to the functions and operations of the various push buttons 18 and knobs 20 and/or voice-enabled commands via microphone 21. Microphone 21 can be any suitable input device for receiving audible inputs. In one embodiment, the display screen may be divided into display screen regions associated with individual buttons 18. This arrangement allows for the indicated function of each button 18 or knob to be readily changed. Additionally, the use of the electronic display screen also permits the buttons and knobs to be labeled in virtually any language.

Surgical console 10 may be adapted for use with a number of different surgical instruments (i.e. surgical peripheral devices 14). For example, these may include a fiber optic illumination instrument, a fragmentation emulsification instrument, a cutting instrument, such as a guillotine cutter for vitrectomy procedures, and micro-scissors inset for proportionate and multiple cutting. While the above-identified surgical instruments are provided for illustrative purposes, it should be understood that the console 10 can be used with other similar equipped instruments.

In general, any surgical instruments that are actuated or controlled by pneumatic or electronic signals may be operably coupled to and controlled by console 10. This control or actuation may be governed by pneumatic, electronic, optical, or other like signals known to those skilled in the art wherein the signals are generated by console 10. Each of these illustrated surgical devices that couple to console 10 may have different modes of operation that may require different settings or parameters that are provided by the microsurgical console. Embodiments of the present invention are adapted to provide audible, spoken word, activation of such control signals that would, in the prior art, be provided by manual input via the control buttons 18 and knobs 20. In this way, the embodiments of the present invention allow the operator, typically a surgeon, to initiate different functions, change settings, turn devices on or off, and otherwise control the functions and operations of surgical console 10 and any peripheral surgical devices connected to surgical console 10 without having to violate the sterility of a surgical field and without the distractions associated with a separate visual and/or tactile control panel.

Embodiments of the present invention provide for a surgical console utilizing user-spoken commands with a pre-defined verbal user/console communication protocol to ensure accurate user-intended surgical console control. The surgical console 10 can comprise microprocessor 11 and memory 13 in addition to microphone 21 and speaker 22. Microprocessor 11 is operable to access and execute software instructions and algorithms stored in memory 13 to parse user-spoken commands received via a command interpreter 15 operably coupled to microphone 21, microprocessor 11 and memory 13. In operation, a user speaks a command to the surgical console 10/microphone 21 and the surgical console 10 provides a command acknowledgement, which can be an audible acknowledgement via speaker 22 or a visual acknowledgement such as a message on screen 17 or a flashing light, and requests a command confirmation from the user. The user confirms the command and the console 10 causes the command to be executed. Voice-activation and control of console 10 and attached devices 14 provides an additional means for surgeons, surgical assistants, scrub techs, and nurses (any user) to adjust console settings, change modes of console operation and otherwise control the operations of console 10 and/or devices 14.

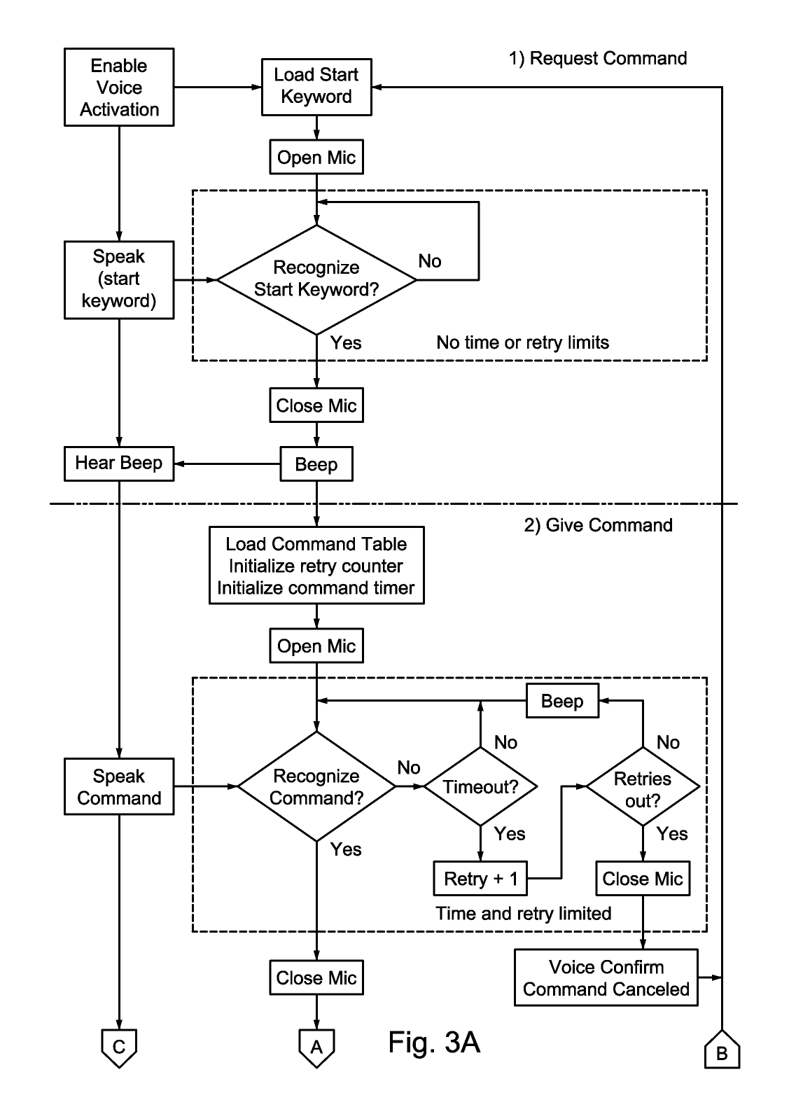

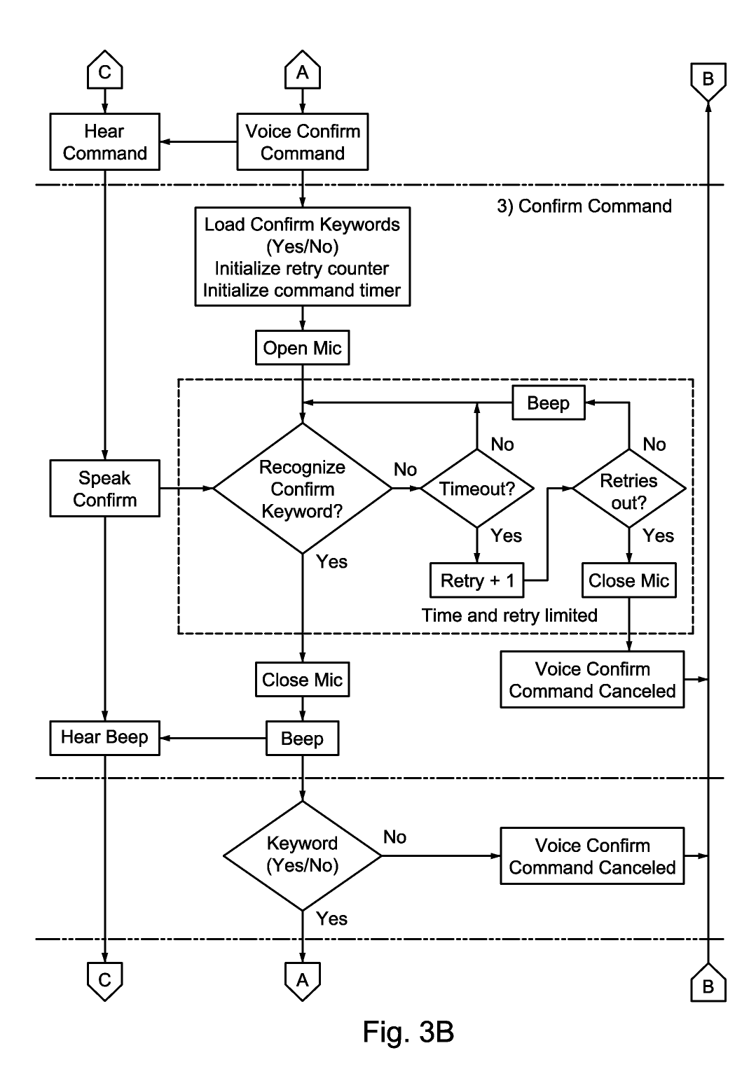

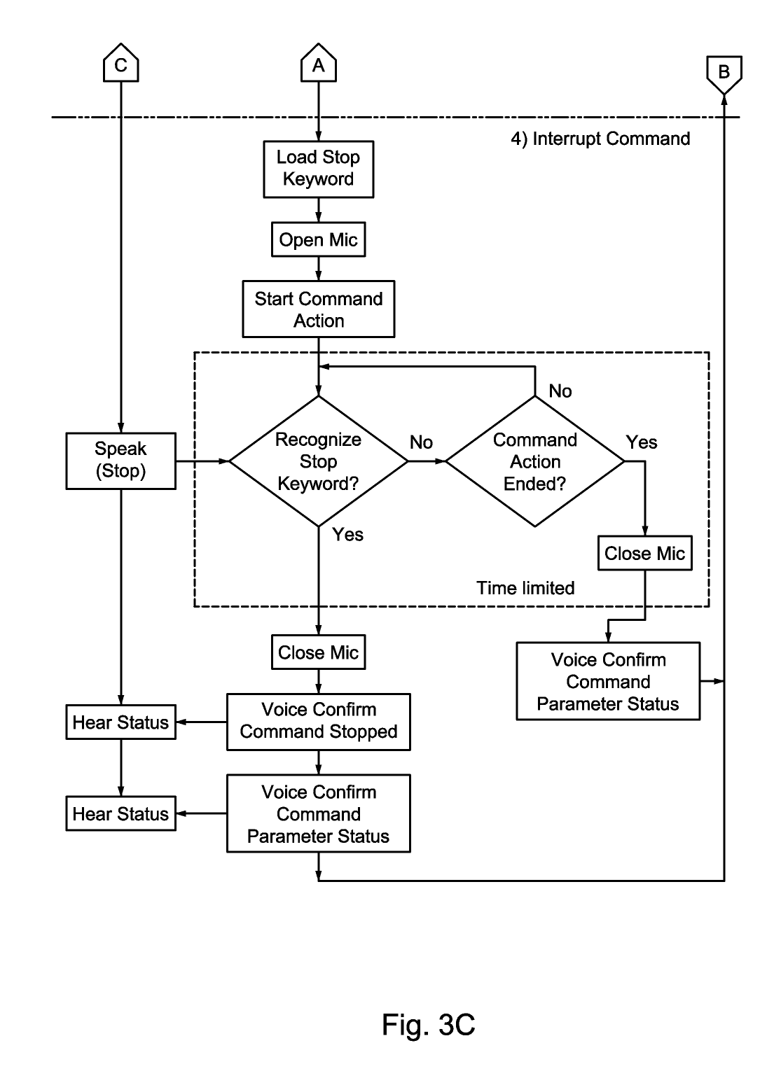

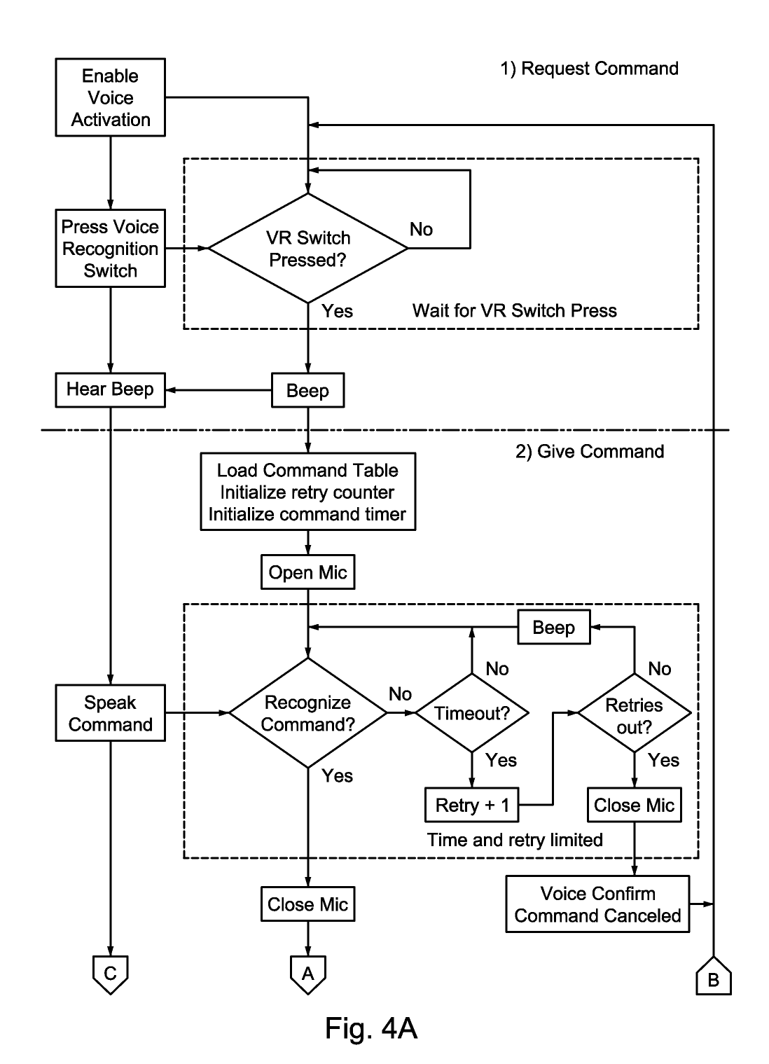

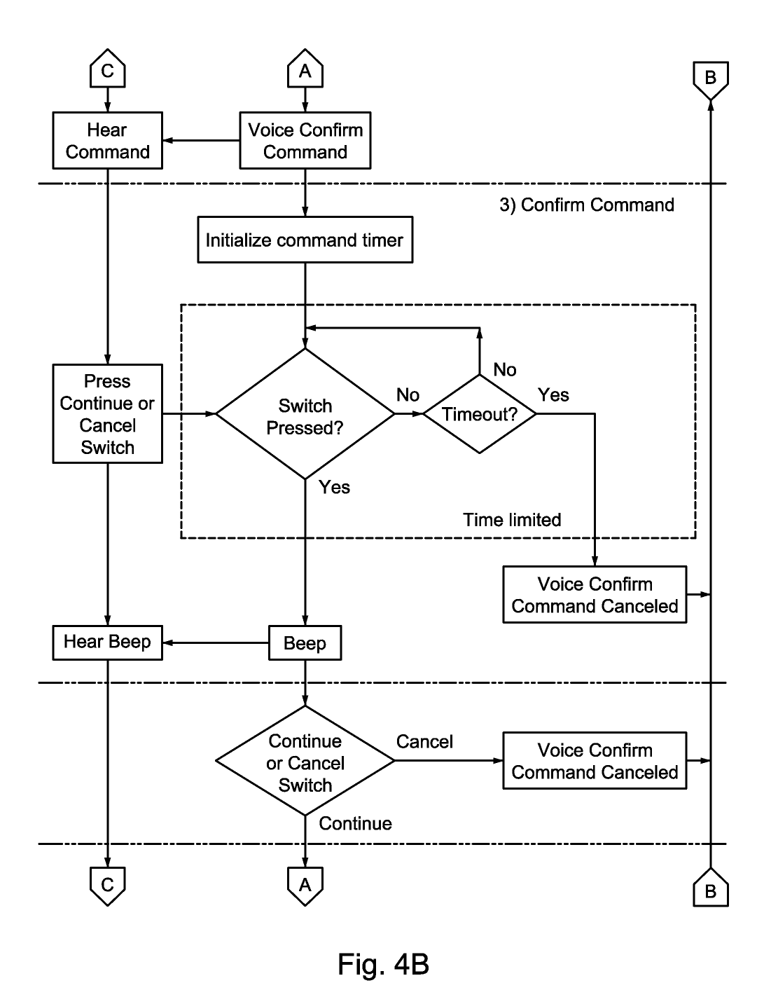

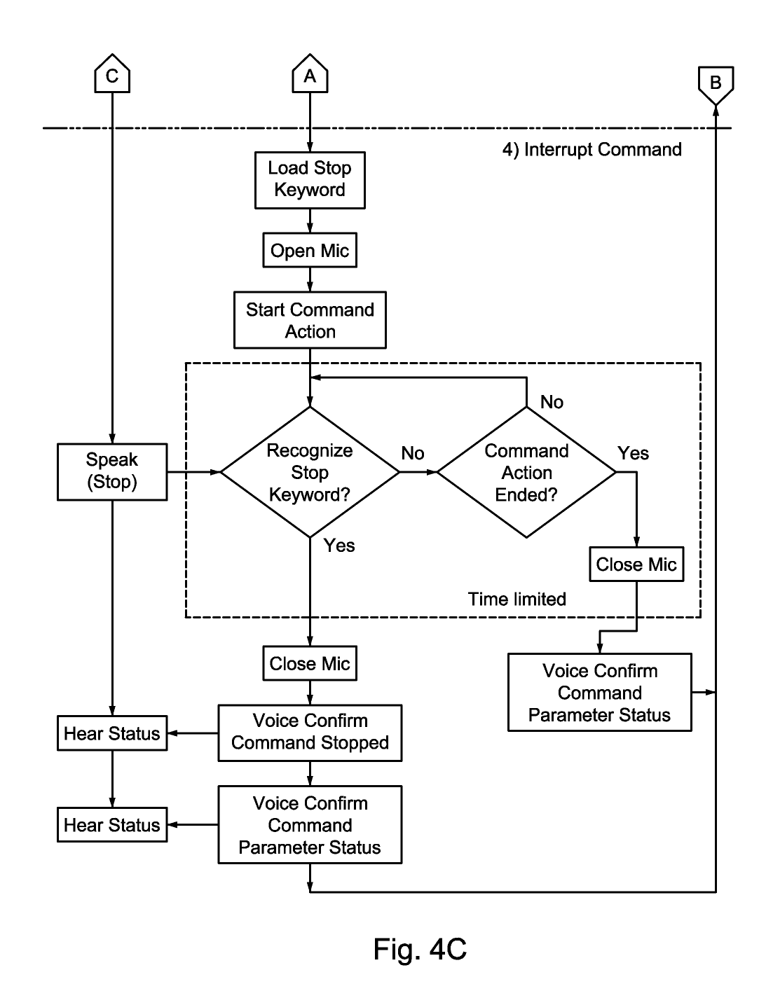

As the operator advances through a surgical procedure, pertinent changes to the operating modes and peripheral device operating parameters are accessed via voice-command and used to initialize or setup the surgical devices 14 for individual steps within an overall surgical procedure. At the completion of a surgical procedure the completed surgical procedure may be saved as a recorded procedure in memory. It should be noted that within surgical console 10, microprocessor 11 couples to memory 13 where the microprocessor is operable to execute the steps that will be discussed in the logic flow diagrams of FIGS. 3A-4C following. At least some of these steps are stored as computer-executable instructions in memory 13.

The microprocessor may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on operational instructions. The memory may be a single memory device or a plurality of memory devices. Such a memory device may be a read-only memory, random access memory, volatile memory, non-volatile memory, static memory, dynamic memory, flash memory, cache memory, and/or any device that stores digital information. Note that when the microprocessor implements one or more of its functions via a state machine, analog circuitry, digital circuitry, and/or logic circuitry, the memory storing the corresponding operational instructions may be embedded within, or external to, the circuitry comprising the state machine, analog circuitry, digital circuitry, and/or logic circuitry. The memory stores, and the microprocessor executes, operational instructions corresponding to at least some of the steps and/or functions illustrated in FIGS. 3A-4C.

Embodiments of the present invention provide advantages over prior art voice-activation and control systems and methods. Embodiments of this invention, unlike the prior art, can recognize and process spoken command phrases rather than simply recognizing multiple combinations of spoken key words, increasing the spoken command recognition accuracy while reducing the likelihood of misunderstood command utterances. Further, embodiments of the present invention require less computer processing power than prior art solutions and the voice-recognition algorithms used are less complicated.

FIGS. 3A, 3B and 3C (collectively FIG. 3) provide a logic-flow diagram illustrating a methodology associated with an embodiment of the present invention. FIG. 3 illustrates the steps associated with one of several voice-recognition algorithms of the embodiments of this invention. The approach of FIG. 3 is that of voice-activation by keyword trigger. In this embodiment, a key word, spoken by a user, establishes a voice-command channel in console 10. This alone can be the activation step for console 10 to receive and process voice-commands, or it can be preceded by a hardware or software interlock that is enabled upon, for example, activation of an on switch to first turn on console 10. Upon system recognition of the key word by console 10 (e.g., microphone 21, command interpreter 15, microprocessor 11, memory 13), which can be indicated by a visual or audible indication at surgical console 10, the user provides a command phrase. Preferably, the command phrase will not contain any non-command phrase words or utterances. The console 10 can then echo the interpreted command for confirmation by the user. Upon confirmation of the command, which is preferably a spoken confirmation, the console 10 causes the command to be executed.

In this embodiment of the present invention the communication protocol is accomplished by a series of four communication segments: request command, provide command, confirm command and interrupt command (optional). Each of these communication segments can further comprise five processes: load key work or command table, open the microphone, recognize the key word or command word or phrase, close the microphone and voice confirm (echo) the interpreted command. These segments and processes are performed by the various components discussed herein of surgical console 10 (microprocessor 11, microphone 21, command interpreter 15, which can be part of microprocessor 11, memory 13, speaker 22, etc.). The interrupt command segment enables a user to cancel a command in progress. This feature will be most useful when a command requires several seconds or more to execute as commands that execute quickly will likely have completed executing before the user can utter the interrupt command.

In another embodiment of the present invention, which can be referred to as voice-activation by switch trigger, a key press initiated by a user establishes the voice-command channel. This embodiment parallels the voice-activation by keyword trigger embodiment described above and is illustrated in the flowchart of FIGS. 4A-4C. In this embodiment, the request command and confirm command processes are performed via key presses (e.g., buttons 18 and/or knobs 20, or via another controller such as a footswitch or remote control) rather than by voice-commands. This embodiment may prove useful for those situations having regulatory and/or safety concerns related to voice-command initiation and confirmation of the command sequence. Additional embodiments may instead perform only one or the other of the request command and confirm command processes with a key press.

Still another embodiment of the present invention, which is referred to here as the voice-activation open microphone embodiment, can comprise constantly monitoring for spoken command phrases via an always active microphone 21. This embodiment, as in all of the embodiments described herein, can also comprise the step of first enabling voice activation via a software, hardware, combination of the two, or other means of activating the system as will be known to those having skill in the art (e.g., voice activation can be configured to be active upon providing power to surgical console 10, or after a software self-test, etc.). In this embodiment, upon recognition of a command phrase via the open microphone 21, the surgical console 10 echoes the interpreted command as described above and upon spoken confirmation of the command by the user, the surgical console 10 causes the command to be executed (i.e., processes associated with the command are caused to be executed by console 10).

For the open microphone embodiment then, the communication protocol is accomplished by a series of three communication segments: provide command, confirm command and interrupt command (optional). The request command segment of the previous embodiments is eliminated by the open microphone. Each of these three segments in turn comprises two processes: recognize the key work, command or phrase and voice confirm (echo) the interpreted command.

It is anticipated that the embodiments of the present invention provide the user the ability to control multiple functions and aspects of surgical console 10 and devices 14 by spoken command. These functions and aspects can be any such functions as will be understood by those having skill in the art and can differ depending on the requirements of the user and the capabilities of surgical console 10. Command phrases and the processes/actions associated with each phrase and that surgical console 10 will cause to be performed upon receiving the command phrase can be programmed and stored within surgical console 10 by well known programming means that will be known to those having skill in the art.

As one of average skill in the art will appreciate, the term “substantially” or “approximately”, as may be used herein, provides an industry-accepted tolerance to its corresponding term. Such an industry-accepted tolerance ranges from less than one percent to twenty percent and corresponds to, but is not limited to, component values, integrated circuit process variations, temperature variations, rise and fall times, and/or thermal noise. As one of average skill in the art will further appreciate, the term “operably coupled”, as may be used herein, includes direct coupling and indirect coupling via another component, element, circuit, or module where, for indirect coupling, the intervening component, element, circuit, or module does not modify the information of a signal but may adjust its current level, voltage level, and/or power level. As one of average skill in the art will also appreciate, inferred coupling (i.e., where one element is coupled to another element by inference) includes direct and indirect coupling between two elements in the same manner as “operably coupled”. As one of average skill in the art will further appreciate, the term “compares favorably”, as may be used herein, indicates that a comparison between two or more elements, items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1.

The present invention has been described by reference to certain preferred embodiments; however, it should be understood that it may be embodied in other specific forms or variations thereof without departing from its spirit or essential characteristics. The embodiments described above are therefore considered to be illustrative in all respects and not restrictive, the scope of the invention being indicated by the appended claims.

What is claimed is:

1. A method of method of operating a surgical console comprising the steps of:
enabling a voice command channel at the surgical console;
receiving a voice command trigger;
receiving a voice command;
confirming the voice command; and
causing the surgical console to execute one or more actions associated with the voice command.

2. The method of claim 1, wherein the actions associated with the voice command comprise a series of surgical steps operable to:
alter operating modes of devices operably coupled to the surgical console; or
alter peripheral device operating parameters of devices operably coupled to the surgical console.

3. The method of claim 2, wherein the surgical console is an ophthalmic surgical console.

4. The method of claim 2, wherein the operating parameters and modes comprise:
pneumatic and electronic parameters associated with microsurgical peripheral devices operably coupled to the surgical console.

5. A surgical console operable to execute spoken commands, comprising:
a microprocessor operable to direct operations of peripheral devices operably coupled to the surgical console;
a memory operably coupled to the microprocessor, wherein the memory is operable to store recorded surgical procedures; and
a user interface, wherein the user interface allows operators to verbally:
initialize the surgical console for a surgical procedure;
select the surgical procedure to be executed;
advance through the steps of the selected surgical procedure,
wherein operating parameters and surgical modes associated with specific steps of the selected surgical procedure are loaded to the surgical console; and
execute the steps of the selected surgical procedure.

6. The surgical console of claim 5, wherein the operating parameters and surgical modes associated with the surgical procedure(s) comprise a series of surgical steps operable to:
alter operating modes of devices operably coupled to the surgical console; or
alter peripheral device operating parameters of devices operably coupled to the surgical console.

7. The surgical console of claim 5, wherein the surgical procedure is an ophthalmic surgical procedure.

8. The surgical console of claim 5, wherein the operating parameters and surgical modes associated with the surgical procedure(s) comprise:
pneumatic and electronic parameters associated with microsurgical peripheral devices operably coupled to the surgical console.
